(19) United States
(12) Patent Application Publication
(10) Pub. No.: US 2011/0294843 A1
(43) Pub. Date: Dec. 1, 2011

(54) ADMINISTRATION SCHEME OF POLAR OPIOID METABOLITES FOR POST-OPERATIVE PAIN MANAGEMENT

(75) Inventors: Mariola S+e,uml o+ee hngen, Aachen (DE); Heimo Stroissnig, Aachen (DE)

(73) Assignee: PAION UK Limited, Cambridge (GB)

(21) Appl. No.: 13/124,476

(22) PCT Filed: Oct. 16, 2008
(86) PCT No.: PCT/EP2008/008773

§ 371 Date: Aug. 15, 2011

(51) Int. Cl. A61K 31/485; A61P 29/00

(52) U.S. Cl. 514/282

(57) ABSTRACT

Administration scheme of an analgesically active polar metabolite of an opioid or the salt thereof for the manufacture of a medicament for the treatment of pain after medical operation, whereas the medicament is administered at least once before the end of the medical operation.

The invention relates to an administration scheme for the treatment of post-operative pain with polar morphine metabolites, in particular morphine-6-glucuronide.

Post-operative pain management is a challenge for patients and physicians. Too large a dose of systemic medication, e.g. an opioid can potentially cause life-threatening respiratory depression, whereas too little a dose can result in intolerable severe pain. Furthermore the under treatment of post-operative pain delays out-of-bed patient mobilization, can prolong the patient's hospital stay, his or her functional recovery, and/or increases monetary loss due to post-operative absences from work.

In contemporary medicine, morphine still is the drug of choice in the treatment of severe post-operative pain. Nevertheless, morphine has several side effects, which can seriously compromise its analgesic effectiveness and the patient safety/compliance.

Among these side effects the post-operative nausea and vomiting (PONV) which is common and distressing is rated by patients as one of the most undesirable complications occurring after medical operations, and may be even more upsetting than post-operative pain. It can be associated with post surgical complications including wound dehiscence, delayed discharge and unplanned readmission.

The occurrence of PONV can range from 17 to 59%, depending on various factors such as type of surgery, anaesthesia technique, gender, the use of antiemetic prophylaxis and/or the use of opioids for post-operative pain relief. Non opioid analgesics are commonly used for their opioid sparing effect to try to reduce this problem.

Several opioids have also been assessed as an alternative to morphine for post-operative analgesia, for example also morphine metabolites, such as morphine-6-glucuronide (M6G). Morphine-6-glucuronide (WHO International Non-proprietary Name: morphine glucuronide, CAS No. 20290-10-2, M6G) is an active metabolite that contributes to morphine's analgesia, particularly in the renally impaired and during long term treatment.

Previous clinical trial revealed that the therapeutic effects of M6G exhibit a later onset, which is probably due to a slower passage of M6G across the blood brain barrier related to its relatively great hydrophilicity (Hanna et al., Anesthiology 2005; 102(4): 815-821).

Thus it is the objective of the present invention to provide a method for treating post-operative pain, which overcomes at least one of the above mentioned drawbacks of known schemes of acute pain management.

This objective is solved by the administration of an analgesically active polar metabolite of an opioid, in particular the morphine metabolite M6G, to the patient for the treatment of post-operative pain at least once before the end of the medical operation.

The core concept underlying the invention is the early administration of the active polar metabolite in order to overcome any delay in the onset of its pharmacological effects, whereas the “early administration” means any time before the end of the operation or at the end of the operation.

According to the invention any analgesically active polar metabolite of an opioid can be used. A preferred metabolite is the glucuronide metabolite, especially morphine-6-glucuronide. However glucuronides of other opioid analgesics such as codeine, levorphanol, hydromorphone, oxymorphone, nalbuphine, buprenorphine, fentanyl, sufentanyl, nalorphine, hydrocodone, oxycodone and butorphanol are also suitable.

A further possible polar metabolite is a sulphate of the opioid, in particular the morphine-6-sulphate. Sulphates of other opioid analgesics, however, can also be administered according to the invention.

The structures of morphine-6-glucuronide and morphine-6-sulphate are shown below:

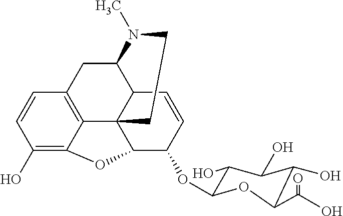

Morphinβ-6-glucuronidβ ((5α,6α)-7,8-Didehydro-4,5-epoxy-3-hydroxy-17-methylmorphinan-β-yl-β-D-glucopyranosiduronic acid)

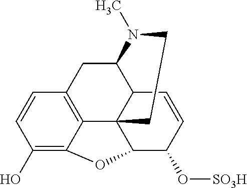

Morphine-6-sulphate ((5α,6α)-7,8-Didehydro-4,5-epoxy-3-hydroxy-17-methylmorphinan-6-yl-sulfate)

Methods for preparation of M6G are described in WO93/03051, WO93/05057, WO 99/58545 and WO9938876. Suitable forms of M6G for use in pharmaceutical compositions are in particular described in EP1 537 132 and EP 0 873 346 B1 with further references as to the manufacture of M6G. All these documents are incorporated by reference as to the disclosure of the manufacture of pharmaceutically acceptable forms of M6G.

The opioid metabolites administered according to the invention can be applied as a free base or as a respective pharmaceutically acceptable salt. Possible pharmaceutically acceptable salts include bromide, chloride and sulphate. In one embodiment of the invention the M6G hydrobromide or M6G sulphate is used. Due to its long term stability, in particular M6G hydrobromide is preferred. Pharmaceutically acceptable salts of M6G, in particular M6G hydrobromide is disclosed in EP 1 537 132 B1, which is incorporated herein by reference.

As stated above, the opioid metabolites according to the invention are polar metabolites. As understood hereinafter the term “polar” means a higher polarity as compared to the unpolar moφhine. The polar nature of a compound can be determined by measuring its partition coefficient between an aqueous buffer and an organic solvent such as octanol. The partition coefficient of morphine, expressed as a logarithm (logP) partitioned between an aqueous buffer and octanol ranges from 0.70 to 1.03 (Hansch C. and Leo A. “Substituent Constants for Correlation Analysis in Chemistry and Biology” Wiley, N.Y., 1979). The polar metabolites of the invention therefore are characterized as having a lower logP value than that of morphine defined above.

The partition coefficient (Log P) of morphine-6-glucuronide measured in a phosphate/n-octanol system has been determined as −2.40. This depends on the specific system. In the literature variation in Log P values can be found measured by different techniques [e.g. −2.95 (Wu D, Kang Y S, Bickel U, Pardridge W M; “Blood-brain barrier permeability to morphine-6-glucuronide is markedly reduced compared with morphine” in Drug Metab Dispos, 1997; 25(6): 768-771.), −1.3 (Gaillard P, Carrupt P-A, Testa B; The conformation-dependent Lipophilicity of Morphine Glucuronides as calculated from their Lipohilicity Potential” in: BioOrganic and Medicinal Chemistry Letters, 1994; 4(5): 737-742) or −0.76 (Avdeef A, Barrett D A, Shaw P N, Knaggs R D, Davis S S; “Octanol-, chloroform-, and propylene glycol dipelargonat-water partitioning of morphine-6-glucuronide and other related opiates”, in: J Med Chem, 1996; 39(22): 4377-4381.). However an overall review of these values indicates that M6G is a polar molecule and significantly more so than morphine.

The term “medical operation” as used herein refers to all kind of medical intervention into the living body, either invasive or non-invasive, for diagnostic and/or therapeutic purposes. “Medical operation” in particular comprises medical treatments which, on a regular basis, are expected to cause post-operative pain for the patient. Thus this term comprises any kind of surgery with anaesthesia, either local or general. It also comprises local interventions, which could be conducted without anaesthesia, for example some endoscopy or other more diagnostic interventions, or e.g. radiation treatment.

As outlined above according to the invention the opioid metabolites are administered to the patient at least once at or before the end of the medical operation”. Most preferred the administration is before the end of the operation. The “end of the medical operation” is the IO point in time, when the intervention at the patient's body is terminated. In case of a surgery it is thus the point in time, when the skin of the patient is closed (“skin closure”). In other procedures, e.g. with radiation, it is the point in time, when the source of radiation is switched off.

In yet another embodiment of the invention the opioid metabolite is administered to the patient at least 10 min, at least 20 min, at least 30 min, at least 45 min or at least 60, 90 or 120 min before the end of the operation. The exact point in time for the administration of the polar opioid metabolites preferably depends of the length and severity of the medical operation.

In a further embodiment of the invention the polar opioid metabolite is administered to the patient even before the start of the operation, i.e. pre-operatively. This is particularly advantageous, if the operation is a minor surgery. A minor surgery is defined for the purpose of this invention as a surgery, which on a regular basis lasts for one hour or less from the induction of anaesthesia until the (planned) end of the surgery.

In other embodiments the administration is performed during the operation, i.e. intra-operatively. Furthermore it is possible to administer the metabolite to the patient together with the induction of the anaesthesia, e.g. concomitantly thereto.

According to the invention the metabolite can be administered at least once before the end of the operation. However it can also be administered more than once, at least two, three or more times. Various administration schemes are possible combining pre, intra- and postoperative administration, whereas each administration step can be singular or multiple.

In a preferred administration scheme the polar metabolite is administered to the patient preoperatively and/or or intra-operatively, whereas one or more doses are given (“loading doses”), followed by a post-operative administration. Post-operatively the metabolite can be administered in one or more doses furthermore followed by one or more individualized doses. The preferred metabolite for this administration scheme is M6G or the salts thereof, in particular M6G hydrobromide.

Post-operatively the polar metabolites can be administered by a patient controlled analgesia (PCA). PCA is a known way of patient's self-administration of analgesia to individualise, and therewith optimise analgesia. This infusion device use by the patient is connected to an intravenous, subcutaneous, ventricular, epidural, or subarachnoid catheter, and narcotic analgesics can be administered by the patient's pressing a button attached to the pump. This device can be programmed to deliver a specified dose of medication on demand at predetermined time intervals to prevent the patient from overdosing or abusing the analgesics.

According to one embodiment of the invention the polar metabolite, in a preferred embodiment M6G or its hydrobromide salt, is administered according to the following administration scheme including at least one step a and b or c below:
a. the medicament is given at least once pre-operatively and/or before the end of medical operation and/or
b. the medicament is given at least once during the post-operative recovery phase (time period between the end of the operation until approximately 6 hours afterwards) and/or
c. the medicament is given afterwards (to a maximum of 48 h) hours after the end of the medical operation) as perfusion, preferably as RCA.

In a specific embodiment steps 1 to 3 are combined.

In one embodiment of the invention in step a above the medicament is administered once or twice, namely about 10 to 40, preferably about 30 min before the end of the operation and/or about 40 to about 80, in particular about 60 min before the end of the operation. Preferably each time a dose of about 5 mg to 15 mg/70 kg body weight, in particular 10 mg/70 kg body weight, is given as an injection over 5 min.

Notably in one embodiment of the invention step a can also be perform without step b and c.

In major surgeries, when anaesthesia is induced by nerve block, the metabolite can be administered in slow i.v. doses starting 150 min after the nerve block.

In a further embodiment of the invention at the end of the operation (at “skin closure”) about 5 to about 50 mg/70 kg body weight, preferably about 10 to about 30 mg/70 kg body weight were administered.

Regarding step b above the metabolite can be administered in about 5 to 20, in particular about 10, about 15 or about 20 mg/70 kg body weight once or twice.

In step c the administration of the metabolite is on demand of the patient, preferably using an infusion of 1 to 3 mg/ml of the metabolite, in particular 2 mg/ml, with a maximum amount of 24 mg/hour.

All of the above embodiments above relating to the specific dosing in particular refer to M6G or the salts thereof, in particular M6G hydrobromide, in an intravenous administration form.

The term “anaesthesia” is defined as lack of normal sensation, especially the awareness of pain, which may be brought on by anesthetic drugs. Anaesthesia is commonly employed prior to surgery so that a patient will not feel any pain or discomfort from incision, tissue manipulation and suturing. Based on the procedure, anesthesia may be provided as local, spinal or general anesthesia. General anaesthesia causes loss of consciousness and can include analgesia, hypnosis, amnesia, Relaxation, and obtundation of reflexes. The local or regional anesthesia causes loss of feeling only to a specified area.

In one embodiment the invention refers in particular to medical operations which are expected to cause moderate or severe post-operative pain. Moderate or severe pain is defined herein as being a pain of at least 4 of the VRS-11 scale.

Pain intensity can be assessed using a Verbal Rating (VRS-11) scale. The VRS-11 scale is an 11-point scale in which the patient is asked to describe their pain as a numeric value over a range of 0=no pain and 10=the worst pain imaginable. A numerical scale was chosen because of the practical difficulties of post-operative patients completing a visual analogue scale. The VRS-11 will be administered by the Patient and the Investigator (observer) by asking the patients to rate their pain using the question: “Please rate your pain at this time from 0-10, with 0=‘no pain’ and 10=‘worst pain imaginable”.

A global assessment of the acceptability of the pain relief provided during treatment can be made using a five-point categorical rating scale as potential answers of the following question:

“How would you rate the pain relief provided after the operation?”
5=excellent
4=good
3=fair
2=poor
1=very poor
   The global pain assessment can be performed by both the Investigator and the patient.

For the assessment of sedation several diagnostic methods are known to the person skilled in art. Sedation can be assessed by using a combination of a binary question and a 3-point ordinal scale. Therefore patients will be asked the following questions:

“Do you feel sleepy?”
Possible answers:
   ‘yes’
   ‘no¹

“if ‘yes’, please rate your sedation:”
Possible answers:
   ‘mild’
   ‘moderate’
   ‘severe’

For the assessment of nausea several diagnostic methods are known to the person skilled in art. Nausea can be assessed using a 4-point ordinal scale. Therefore patients will be asked the following question: “Would you describe your experience of nausea (feeling sick) now as none, mild, moderate or severe?”

For the assessment of vomiting/retching several diagnostic methods are known to the person skilled in art. The occurrence of vomiting/retching can be assessed by asking the patient the following question:

“Have you retched or vomited since the last time I asked?”
Possible answers:
   ‘yes’
   ‘no

“If ‘yes’, please specify the number of times since the last assessment.

### EXAMPLE 1

### Comparison of the Analgesic Efficacy and Tolerability of Morphine-6-Glucuronide and Morphine in Patients Following Major Abdominal Surgery.

BACKGROUND: The M6G022 study was a randomized, placebo-controlled, double-blind study that investigated the tolerability and analgesic efficacy of morphine-6-glucuronide, M6G and morphine in patients following major abdominal surgery. A schematic outline of the study design is given in FIG. 1A. A total of 517 patients undergoing abdominal hysterectomy, bowel or gastrointestinal surgery, or major urological surgery were randomised to receive either M6G or morphine for the relief of moderate to severe post-operative pain. Pain relief, nausea and emesis were recorded for 24 to 48 hours after start of the patient controlled analgesia.

METHODS: Prior to the operation, patients were randomised to receive either M6G or morphine using a schedule prepared by an external agency. The study site personnel (with the exception of the pharmacy); representatives of the sponsoring company; and the organisation involved in monitoring, data management, or other aspects of the study were blinded to the study treatment. The use of midazolam, temazepam, zopiclone or other short-acting pre-operative anxiolytics was allowed. All patients received a standardised general anaesthetic consisting of propofol induction, with either isoflurane or sevoflurane maintenance. These inhalational agents are associated with a similar risk of PONV. Nitrous oxide was not allowed because of its emetic effects, and the risk of an imbalance in its use. An initial bolus of 1-3 μg/kg of fentanyl or 0.1-0.3 μg/kg of sufentanyl followed by further doses as required, was used for pre- and peri-operative analgesia. Post-operative analgesia was provided by the study drug. A loading dose (a standard 10 mg/70 kg of morphine or 30 mg/70 kg of M6G) was administered as an intravenous injection over 5 minutes, 30 to 60 minutes prior to the end of surgery. The loading dose of M6G was based on prior experimental human and clinical studies showing morphine and M6G to have a potency ratio of about 3 to 1 and has been confirmed in a larger study. During recovery in a post-anaesthetic care unit, patients could receive one or two further doses of study drug (15 mg/70 kg M6G, or 5 mg/70 kg morphine, over 5 minutes) to achieve a baseline pain rating of ≦3 on an investigator administered eleven point Verbal Rating Scale (VRS-11) where 0=no pain and 10=worst pain imaginable. Patients who did not achieve this were withdrawn from the trial at this point as it was felt unethical to withhold for longer from patients in pain analgesics known to be clinically effective.

When comfort had been achieved, patients could self administer study drug via a PCA delivery system (Graseby 3300, Smiths Medical, Watford, UK) as required to maintain pain relief. Each effective demand delivered 1 ml of a 2 mg/ml solution of M6G, or 1 ml of a 1 mg/ml solution of morphine, with a 5 minute lockout period between each administration. The ratio of M6G to morphine of 2:1 was chosen on the basis of results from earlier pilot studies in the drug's development programme. These suggested a lower dosage requirement for maintenance of analgesia once adequate levels had been achieved by administration of loading doses, because of the agent's slower elimination from the brain. The investigator could administer up to 5 ml of PCA study drug, on no more than two occasions in each 24 hour post-operative period, as rescue medication if the patient was in uncontrolled pain. Additionally all patients received 1 g paracetamol orally or rectally every 6 hours as a morphine sparing agent. The patients stayed on PCA for a minimum of 24 hours following surgery, but could continue up to 48 hours if clinically indicated.

No prophylactic antiemetic was given, although iv ondansetron (4 mg/2 ml) was available if the patient experienced severe nausea, or immediately following emesis. If this was ineffective further doses of ondansetron; or other agents such as cyclizine, prochlorperazine or perphenazine; were allowed.

Pain intensity at rest was assessed by the patients using the VRS-11 scale at baseline, 15, 30 and 45 minutes and 1, 2, 4, 6, 9, 12, 15, 18, 21 and 24 hours after the start of PCA. A numerical scale was chosen because of the practical difficulty for post-operative patients of completing a visual analogue scale. If the patient remained on PCA after 24 hours, the pain scores were recorded every 8 hours up to 48 hours. In addition, at the end of the study the patients and observers were asked to assess the acceptability of their pain relief over the entire study using a 5 point rating global scale ranging from very poor (1) to excellent (5). The number of times each patient pressed the PCA button to receive study drug was monitored throughout the study, including both “good demands” (when study drug was received) and “bad” (when study drug was not received due to the 5 minute lock out period). The total consumption of study drugs was calculated.

Emesis risk factors of female gender; history of motion sickness or PONV; or smoking status were recorded at screening. Post-operative nausea was assessed by the patient using a 4 point ordinal scale (none, mild, moderate, severe) and occurrence of vomiting or dry retching recorded (yes/no; and if yes, the number of episodes) pre-operatively (Day −1), at baseline, and at 1, 2, 4, 6, 9, 12, 15, 18, 21, 24, 32, 40 and 48 hours after administration of the test analgesic. Nausea and vomiting were also combined in a single scoring system (postoperative nausea & vomiting, PONV), with an episode of vomiting or dry retching being scored as a 3 (severe) on the nausea scale.

Sedation was assessed using a combination of a binary question (sleepy, yes or no) and a 3 point ordinal scale (mild, moderate, severe) if the patient was sedated. All adverse events were recorded, along with concomitant medication use, with particular attention being paid to the use of anti-emetics.

Statistical Analysis

The Intention to Treat (ITT) set of patients comprised all those who had been exposed to study medication at least once. This analysis set was the basis for the main efficacy and all safety and demographic analyses. Data were double entered using interactive verification into a CLINTRIAL database (Version 3.3.3, Phase Forward, Waltham, Mass., USA), and analysed using SAS (version 8.2, SAS Institute Inc., Cary, N.C., USA).

The trial's primary objectives were to compare the incidence and severity of nausea from M6G and morphine, following confirmation of non-inferiority in pain relief from the doses given. The primary endpoints were (i) pain relief over the 24 hour post-operative period, as assessed by the AUCO-24 of pain intensity using VRS-11, and (ii) the incidence and severity of nausea 6 to 24 hours after achieving baseline relief, determined using AUC6-24 of the nausea verbal rating scale score. In designing the study the time period of 6-24 h had been chosen to minimise the effect of the general anaesthetic regimen on PONV and provide the clearest picture of opioid related emesis. The size of the study was calculated using data from Sanansilp with the expectation of a 25% reduction in the M6G group in the percentage of patients suffering from nausea in the morphine group (odds ratio of 1.8). A sample size of 410 patients (205 in each treatment group) would have had 90% power to reject the hull hypothesis. Non-inferiority was established if AUCO-24 of pain scores for M6G was not more than 10 mm per hour higher than that for morphine, with 97.5% confidence (one-sided interval). Once non-inferiority in pain relief was established, superiority comparing levels of nausea was tested at the 5% level. The hypotheses for both endpoints were tested using an ANCOVA model including terms for treatment, surgery type and pooled centre. The “last observation carried forward” technique was used to include in the analysis the pain data of patients who withdrew from the study early. The patients who withdrew before 6 h did not contribute to the assessment of PONV₁ and there was an imbalance in the numbers on M6G and morphine who were withdrawn, especially because of inadequate pain relief in the early stages. In case inclusion of these patients in the analysis might have affected the primary outcome a sensitivity analysis was undertaken. For all patients who were withdrawn prior to baseline, and any who were not withdrawn but for whom no AUC had been calculated because of missing data, simulated nausea profiles were derived using the mean, median, adjusted mean, mean+1SD and mean−1SD values for the treatment groups at each timepoint. The main analysis was then rerun with the withdrawn patients included using data derived from each of these simulations.

Results:

Five hundred and seventeen patients were enrolled into the study; 268 randomised to receive M6G and 249 morphine. The treatment groups were well matched with respect to age and type of surgery; and nausea risk factors. Patient demographics are shown in Table 1. A schematic outline of the drug administration regime of the study is given in FIG. 1A, the patient disposition is summarised in FIG. 1B. Two hundred and twenty three patients (83.2%) receiving M6G and 227 patients (91.2%) receiving morphine completed 48 hours on PCA (FIG. 1B).
Use of non-study intra-operative opiates was balanced between the two treatment groups, with 176/268 (65.7%) of those in the M6G group receiving fentanyl and 91/268 (34.0%) receiving sufentanil. Corresponding figures for the morphine group were 166/249 (66.7%) and 83/249 (33.4%) respectively.

M6G shows a trend to better analgesia in the time window from 6 h to 24 h. The difference between the adjusted means of pain intensity AUCs for M6G and morphine over the 24 hours was +3.318 (95% CI: −4.017, 10.653), showing that M6G was non-inferior to morphine with respect to pain intensity in the whole 24 hour period. However during the late period 6-24 hours the difference in pain AUC showed a trend in favour of M6G over morphine (FIG. 2A).

Patients demanded less M6G in comparison with morphine in the time window from 4 hours to 24 hours.

To achieve comfort initially patients required more M6G (45.0 mg/70 kg) than morphine (14.4 mg/70 kg) approximately in the expected ratio of 3:1. However during the first 24 hour period of PCA the ratio was 1.7:1, and was even less (1.3:1) during the second 24 h of PCA (in those patients who continued for this period). PCA demands in both study groups were more numerous during the early post-operative period (up to 4 hours) (FIG. 2B). Patients in the M6G arm demanded further analgesia at greater frequency to those on morphine at this time (mean totals of demands for this period M6G 33.5, morphine 28.8; p=0.227), whereas patients in the M6G group demanded analgesia less frequently during the later stages of the study (mean totals for 4-48 h M6G 53.1, morphine 93.9; p=0.004).

The incidence and severity of the combined measure PONV was reduced significantly in patients receiving, M6G. The incidence and severity of the combined measure PONV showed reductions in favour of M6G for all time periods assessed, varying between 24.3% and 28.5% (FIG. 3B, Table 2), and these were statistically significant for periods up to 24 hours. Results of a sensitivity analysis were similar to those for the nausea score AUCs.

The incidence of the combined measure PONV was reduced significantly in patients receiving M6G.

Similarly, comparison of the AUCs for vomiting and dry retching for the first 24 hours showed a 34.2% reduction in favour of M6G from 6-24 hours (p=0.047), and a 32.3% reduction from 0-24 hours (p=0.044) (FIG. 3B).

Patients receiving M6G required less antiemetic medications. There was a significant reduction in antiemetic use for the M6G patients, with 35.4% requiring anti-emetic medication, compared with 44.2% on morphine (p=0.043).

Patients receiving M6G showed less sedation.

At the point when the patients were given control of the PCA button (baseline) most were sedated (203/268 M6G [75.7%]; 219/249 morphine [88.0%], p=0.016). The sedation score of 71 patients 56 (20.9%) on M6G and (28.5%) on morphine was recorded as severe at this point. Mean sedation scores for M6G were lower than for morphine (FIG. 4); and the differences were statistically significant at baseline 1, 2 and 4 hours.

Conclusions:

After administration of M6G most patients received good analgesia during the early postoperative period; and most rated their overall post-operative pain relief as excellent or good. In comparison to the less polar opate morphine, M6G exhibited a slower onset time with relatively weaker analgesia within the first hour postoperatively. This slower onset could be overcome by an earlier administration of the loading dose of M6G, to achieve full early postoperative analgesia.
At doses shown to be equianalgesic, the incidence and severity of nausea and vomiting after the commencement of PCA were higher in patients receiving morphine than those receiving M6G, despite higher use of antiemetics in those with severe symptoms. The reduction in risk of nausea was greater in those patients with the known risk factor of female gender, with a trend to a larger reduction in those with the other risk factors. The study also included a combined measure of nausea and emesis (PONV), which also showed statistically significant reductions for M6G treatment compared to morphine (28.5% between 6 and 24 hours, p=0.018). The magnitude of reduction of PONV over the initial 24 hours post-operatively. in the region of 24-29%, is similar to the effect of ondansetron¹⁰. Whilst the main purpose of the study was to assess PONV, the observed reduction in sedation with M6G compared to morphine was of interest. Severe sedation is clinically important in post-operative management as it predisposes to respiratory depression and hypoxaemia. Due to the lesser propensity of M6G to cause sedation it should have a lower potential than morphine for inducing respiratory depression.

### EXAMPLE 2

Meta Analysis of Clinical Studies with M6G Pain Treatment after Surgery

Background & Methods:

This meta analysis is based on four CENES-sponsored M6G studies with data of 769 patients, whereby 446 patients received M6G and 323 patients received morphine (see Table 3).

The four studies M6G001, M6G012, M6G015 and M6G022 are described in the following.

M6G001A: The aim of this proof of concept study was to select a therapeutic dose of M6G when compared with a standard dose of morphine in patients requiring post-operative analgesia following hip-replacement surgery (n=18). This was a double-blind, randomised study. Three dose levels of M6G (10, 15, and 20 mg/70 kg) or 1 dose level of morphine (10 mg/70 kg) were administered as IV bolus injections followed by PCA with M6G or morphine for 24 hours.

A therapeutically equivalent dose of M6G to morphine could not be determined in this study since PCA was required early after surgery by the majority of patients. All doses of M6G were found to be well tolerated.

M6G012: The aim of this study was to compare the analgesic effect of two different dose regimens of M6G with a standard regimen of morphine on acute pain following hip-replacement surgery under a general anaesthetic (n=68). Patients were randomised to receive one of three IV treatment regimens (placebo at induction of anaesthesia+10 mg/70 kg morphine at skin closure, placebo at induction of anaesthesia+30 mg/70 kg M6G at skin closure, or 20 mg/70 kg M6G at induction of anaesthesia+placebo at skin closure). Patients were allowed post-operative rescue with morphine, and morphine was also administered post-operatively to all treatment groups via a PCA device.

Over the four hour post-operative period, 30 mg/70 kg M6G administered at skin closure was non-inferior to 10 mg/70 kg morphine administered at skin closure when pain was assessed using a VAS. Patient assessment of overall pain relief provided by the morphine and M6G treatment regimens over the 24 hour post-operative period was similar.

M6G015: The aim of this randomised, placebo-controlled, double-blind, dose-ranging study was to compare the consumption of morphine as rescue and PCA medication during the 12 hours following the administration of one of three different slow IV doses (10, 20, and 30 mg/70 kg) of M6G or placebo in patients suffering pain following total knee-replacement surgery under a standard spinal nerve block (n=170). Patients received a single dose of study treatment 150 minutes after the administration of the nerve block. This was followed by morphine administered as rescue medication and PCA. Efficacy was evaluated by monitoring the use of post-operative rescue medication (morphine) delivered through a PCA device over the 24 hour post-operative period, by assessment of pain using a 100 mm VAS, and by a global pain assessment. The complete study has recently been reported by Dahan et al. (Eur J Pain, 2008; 12(4): 403-411).

In general, the results for patients' overall opinion of pain relief provided by study treatment showed that the majority of patients rated pain relief given by the study treatment as “good” or “excellent”. There were no statistically significant differences between the patients' opinion of pain relief provided by the study treatment between any of the M6G treatment groups compared with the placebo treatment group, which was to be expected since patients were able to use PCA as required.

M6G022: The design of the study together with the results are presented as example 1 of the invention.

Meta Analysis

The following variables were included in the meta analysis:

Nausea (none, mild, moderate, severe)

Vomiting (yes/no)

Sedation (none, mild, moderate, severe)

Pain (none, mild, moderate, severe)

Respiration rates

ICU/Recovery time

Anti-emetic medication

The initial results of the meta analysis revealed a statistically significant difference for nausea, sedation and vomiting (see Results). Therefore additional subanalyses were performed with regard to the Stratification according to treatment (morphine only, M6G only, combination of morphine and M6G) and the assessment of analgesia separated for 0-6 hours, and 6-24 hours.

Results:

The severity of post-operative nausea was reduced significantly in patients receiving M6G.
A moderate to severe nausea was observed in 23.9% of the M6G-treated patients and 32.8% of the morphine-treated patients (FIG. 5). This difference is significant (p<0.0248).

The incidence and severity of post-operative sedation was reduced significantly in patients receiving M6G.

A moderate to severe sedation was observed in 63.5% of the M6G-treated patients and 81.4% of the morphine-treated patients. This difference is highly significant (p<0.0001). 5

The incidence of post-operative vomiting was reduced significantly in patients receiving M6G.

Vomiting was observed in 26.0% of the M6G-treated patients and 36.5% of the morphine-treated patients (FIG. 6). This difference is significant (p<0.0102).

The analgesic efficacy of M6G is superior to morphine in the time window of 6 to 24 hours.

In the time window from 0 to 24 hours after start of the PCA there was no statistical difference between the analgesic efficacy of morphine and M6G (see FIG. 7A). However, the assessment of analgesia for the time window of 6-24 hours showed that M6G is superior to morphine. In this time window a moderate to severe pain was observed in 19.1% of the M6G-treated patients and 34.0% of the morphine-treated patients (see FIG. 7C). In the complementary time window ranging from 0 to θhours there was no statistical difference between the analgesic efficacy of morphine (54.2%) and M6G (50.5%) (see FIG. 7B). This meta analysis is in accordance with the analysis of the M6G022 study showing that M6G has a slower onset of action.

The pain-free time during first 24 hours was prolonged in patients receiving M6G.

For patients that received morphine, a pain-free time from 9.0-10.9 hours was recorded, 15 whereas the M6G-treated patients showed a prolonged pain free time from 11.6 hours to 14.5.

### EXAMPLE 3

Pharmacokinetics of Morphine-6-Glucuronide at Single and Multiple Analgesic Doses

### BACKGROUND

Morphine-6-glucuronide (M6G) is a minor (10%) metabolite of morphine, with comparable affinity for the μ-opioid receptor. As it is significantly more polar than morphine and its penetration of the CNS relatively poor, it does not contribute to the activity of single doses of i5 morphine. However, during chronic administration of morphine, M6G can accumulate in the CNS and prolong analgesia. Oral doses of M6G are poorly absorbed, but a 30 mg intravenous dose is as effective an analgesic as 10 mg morphine for severe pain. The pharmacokinetics (PK) of M6G as a metabolite of morphine have been studied on many occasions as have its PK when given at low intravenous doses. This example describes the PK of the compound when given intravenously to healthy volunteers at single and multiple clinically-relevant doses.

Material & Methods

Healthy male and female subjects were divided into two groups (A and B) with 12 in each. Group A received three single intravenous infusions of M6G over 5 mins at levels of 15, 30 and 45 mg/70 kg in random order, having taken 50 mg of naloxone orally 24 hrs beforehand. There was a seven-day wash-out period between each dose. Blood samples were taken pre-dose and at the following time-points: 3, 5, 15, 30 mins and 1, 2, 3, 4, 6, 12 and 24 hrs after the start of the infusion. Group, B received five, 15 mg/70 kg, intravenous infusions of M6G, each over five minutes, at 6-hourly intervals, having taken a 50 mg oral dose of naloxone 24 hrs before the first infusion. Blood samples were taken before each of the 5 doses, and at 3, 5, 15, 30 mins and at 1, 2, 3 and 4 hrs after the initiation of the 1st and 5th infusions and at 6 hrs after the 5th dose. Vital signs (supine blood pressure, pulse, respiratory rate and oral temperature), respiration rate, pulse oximetry and adverse events were monitored during the study. Plasma levels of M6G were measured using HPLC/ms/ms.

Results:

Single dose: Some PK parameters for M6G after a single 30 mg intravenous dose are shown (Table 4). The dose-proportionality of exposure to M6G was assessed by comparison of the three doses in Group A. Exposure, expressed as Cmax, AUCO-t and AUCO-oo, increased in a directly dose-proportional manner following 15, 30 and 45 mg (single dose) intravenous infusions of M6G (Table 5).

Multiple dose: Accumulation of M6G, calculated by comparing AUC(0-6 h) after Doses 1 and 5 in Group B, was approximately 8%. Steady-state was achieved by the fifth dose and the trough levels of M6G (63.9 16.5 ng/ml) were consistent.

Conclusions:

Exposure, expressed as Cmax, AUCO-t and AUCO-∞, increased in a directly dose-proportional manner following 15, 30 and 45 mg (single dose) intravenous infusions of M6G. Steady state was shown to have been achieved within five 6-hourly intravenous infusions of 15 mg M6G administered at 6 hour intervals, by comparison of AUCO-6 after Dose 5 and AUCO-∞ after Dose 1. Average peak concentrations of M6G were approximately 25% higher following Dose 5 than Dose 1. Trough levels of M6G, measured 6 hours after each dose, were consistent, averaging 63.9±16.5 ng/ml. There was little accumulation in terms of AUC: 8.1% for AUC0-6 and 2.9% for AUCO-oo.
No clinically significant safety issues were identified, the nature of the adverse events being comparable to those reported historically with morphine therapy.

### EXAMPLE 4

### Pharmacokineti/Pharmacodynamic (PK/PD) Modelling for M6G

Background & Methods:

PK/PD modelling is based on PK data for M6G from population meta-analysis of 150 5 subjects in 6 studies including 2545 plasma concentrations (Table 7).

PK data for morphine are derived from 18 subjects in 1 study including 193 plasma concentrations. In addition data from two published population PK studies were included.

A “three compartment model” was applied to simulate PK parameters for M6G and Morphine by using the software “NONMEM” (version 6).

A “Monte Carlo simulation” was applied to simulate the pharmacodynamics for M6G and Morphine by using the software “ModelMaker” (version 4).

Results:

General analysis of PK data:

The analysis revealed that the systemic clearance of M6G can be predicted from renal function, the systemic clearance of M6G is reduced to about half in anaesthetized patients and the body weight is a good predictor of central and peripheral volumes.

For morphine no clear predictors of the pharmacokinetic parameters could be found.

Assessment of the PK/PD model performance

In order to assess the goodness of the model the weighted residual (WRES) were analysed, which show the difference between the patient's value and the predicted value. There should 0.5 be roughly equal numbers of positive and negative values and any large numbers might be outliers. With NONMEM-weighted residuals like these, a value of + or −6 suggests an outlier.

The Goodness of Fit plots revealed that the vast majority of the WRES values are in the JO range of −2 to +2, and only a few excess positive numbers could observed (FIGS. 11 & 12). In sum, the analysis verify the goodness of the established PK/PD model. As a result the PK/PD modeling should allow the simulation of the analgesic efficacy and the side effects (esp. PONV) after i.v. administration of different doses of morphine and M6G.

The simulation shows a slower onset of M6G vs. Morphine.

The comparison of the simulated pain scores after i.v. administration of 15 mg morphine and 30 mg M6G revealed that M6G shoes a slower onset of analgesic activity (FIG. 13). It is important to note that at later time points the M6G is superior to morphine, which is also in agreement with the results of the clinical studies. LO

Furthermore, this simulation nicely demonstrates that M6G produces a long-lasting analgesia that was also evident in the clinical studies.

M6G given 2 hours earlier than morphine is superior to morphine throughout the whole time period

In order to obtain a M6G dose and administration scheme that is superior to morphine several simulations were performed. As a result it was found that an earlier administration of 45 mg M6G as compared to 15 mg morphine resulted in a superior analgesic activity over the whole time period (FIG. 14).

Subsequent M6G doses of 15 mg could maintain the analgesic activity. A further simulation demonstrated the effect of subsequent M6G administration at a dose of 15 mg with time intervals of 6, 8 and 12 hours. With time intervals of 6 or 8 hours the analgesic activity was nicely maintained, but also a time interval of 12 hours significantly attenuated the loss of analgesic activity of the starting dose (FIG. 15). These results are in good agreement with the results of the human PK study (Example 3) where the administration of 15 mg at six hour intervals resulted in a steady state of the drug in the plasma.

M6G given at different doses of show less side effects (PONV) compared to morphine.

A simulation of the side effects (PONV) revealed that M&G at doses of 30 mg and 45 mg induced considerably less side effect than 15 mg of morphine (FIG. 16).

M6G exhibits a greater therapeutic window than morphine

The comparison of the simulated pain scores and side effects (PONV) for several doses of morphine and M6G allowed the calculation of ED50 values for the analgesic activity and the side effects (Table 9, see also Table 8). The therapeutic window as exemplified by the ratio between the pain-related ED₅₀ and the side-effect related ED₅₀ is 5 times greater for M6G than for morphine (M6G: 7.5 vs. Morphine: 1.5; see Table 9 right column).

### List of Tables

Table 1: Patient demographics of the M6G022 study

Table 2: Post-operative Nausea and Vomiting observed in the MΘG022 study

Table 3. M6G studies used for Meta-Analysis

Table 4: PK parameters for M6G after 5 min infusion at 15, 30 and 45 mg/kg to healthy volunteers.

Table 5: Statistical analysis of dose proportionality after single 5 min infusion of M6G at 15, 30 and 45 mg/kg.

Table 6. Summary of the M6G multiple dose pharmacokinetic parameters following the first and fifth dose of 15 mg/kg M6G to healthy volunteers.

Table 7 Overview on data used for the PK/PD modeling

Table 8 Comparison of 24-Hour Pain AACs and PONV AUCs for different doses of M6G and Morphine using median PK values.

Table 9 Calculation of therapeutic margin

### LIST OF FIGURES

FIG. 1: (A) Schematic outline of the study procedure and (B) Patient Disposition of the M6G022 study.

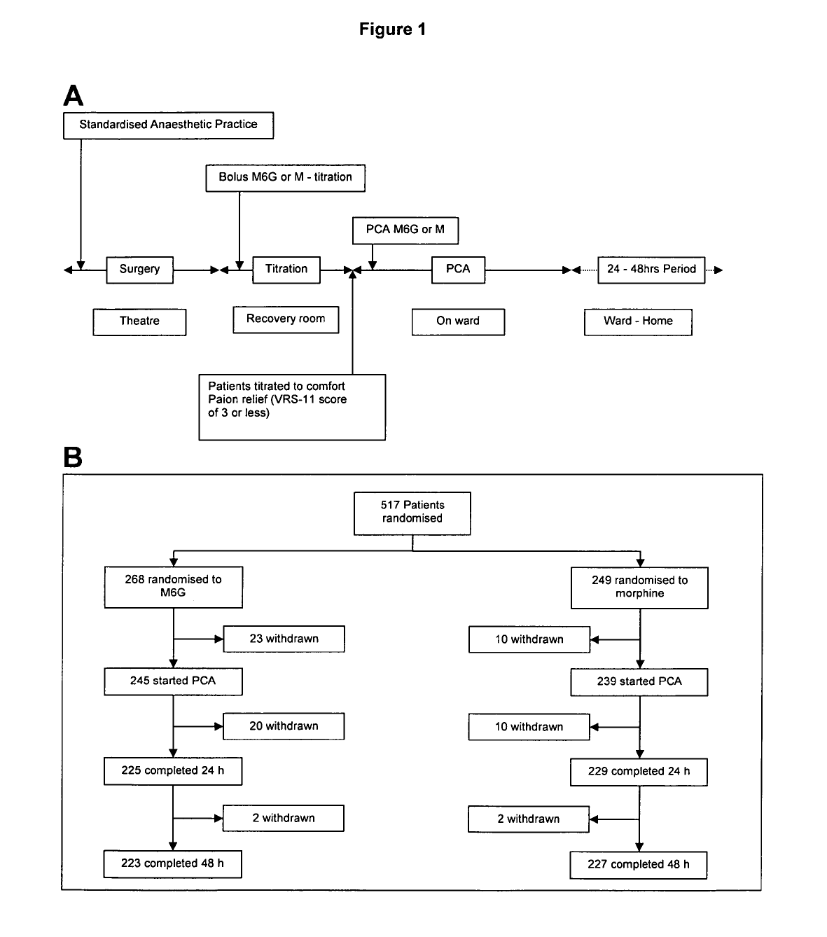

FIG. 2: Pain scores and total PCA demands in the morphine and morphine-6-glucuronide treated groups. A. Pain intensity during 24 h of PCA (Inset 0-6 hours). Pain intensity is measured on an 11 point Verbal rating Scale (VRS-11). B. Numbers of PCA demands per hour. Values are mean+/−SEM.

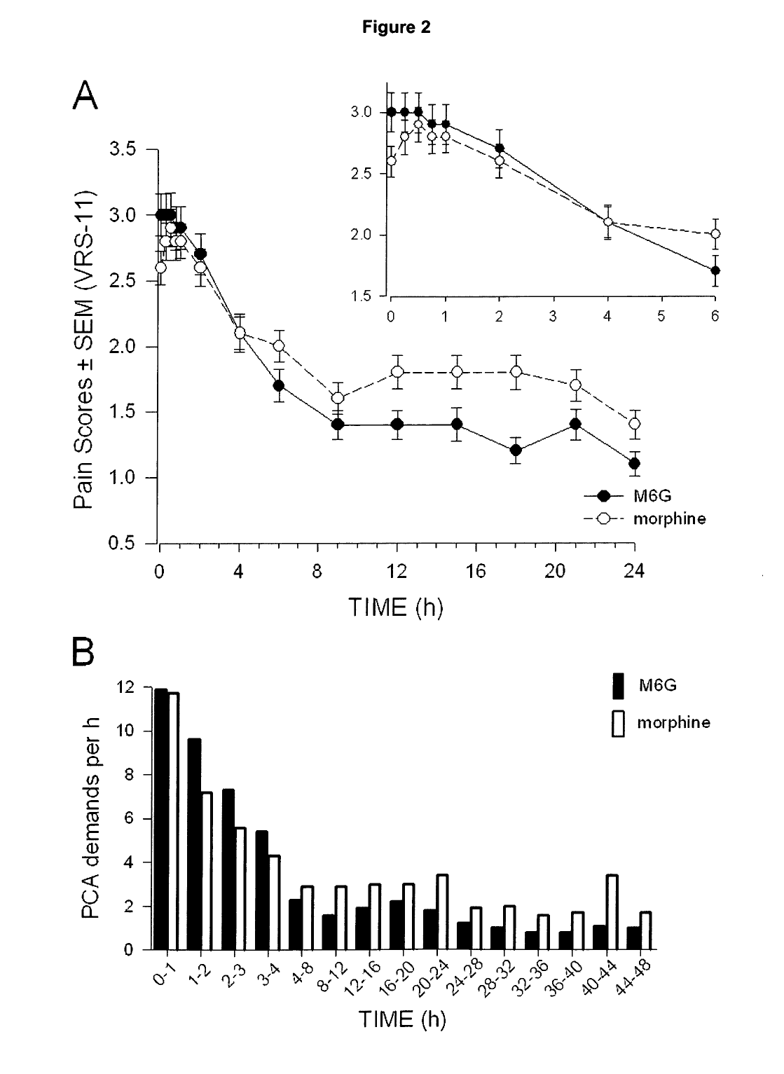

FIG. 3: Nausea, retching and vomiting in the morphine and morphine-6-glucuronide treated groups. A. Nausea severity as measured using a 4-point numerical scale during the first 24 h of PCA. B. Area under the Curve of Scores for Nausea, Retching/Vomiting and PONV for the first and the second 24 h on PCA. Values are mean+/−SEM.

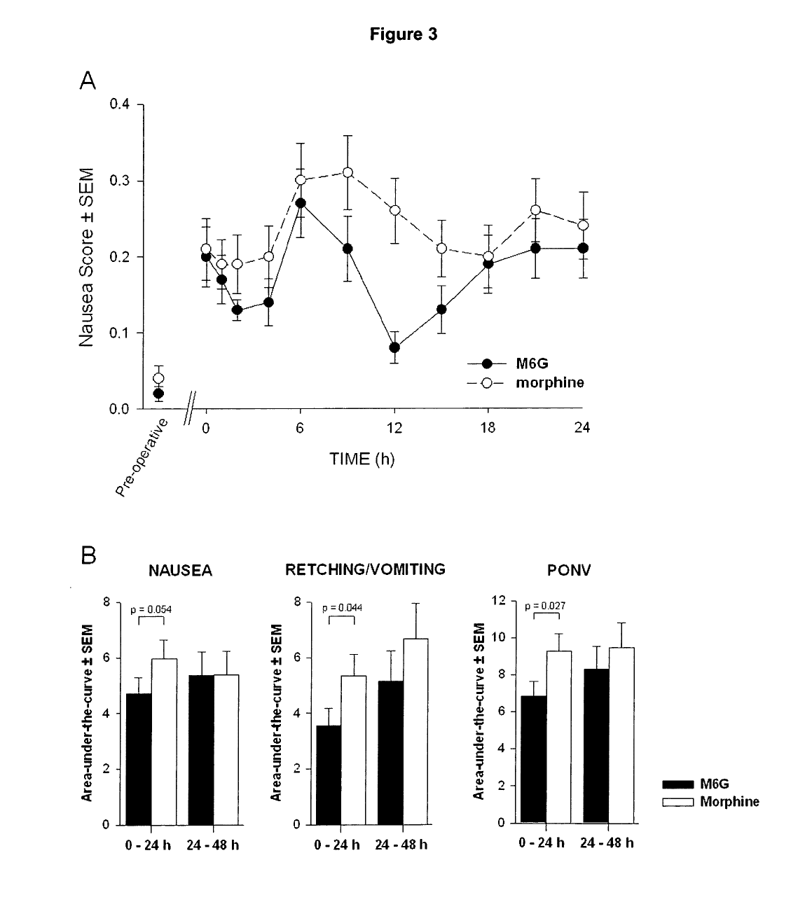

FIG. 4: Sedation scores in the morphine and morphine-6-glucuronide treated groups as measured on 3 point numerical scale for the first 12 h of PCA, *p<0.01**p<0.05.

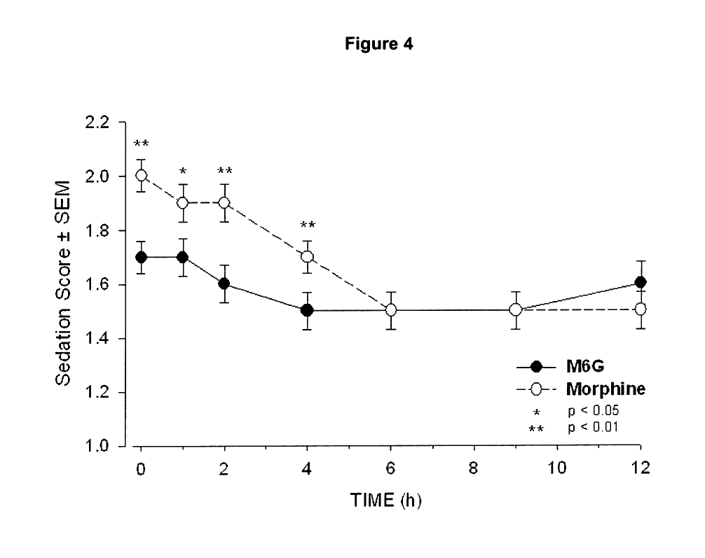

FIG. 5: Worst assessment of nausea in the meta analysis of M6G clinical studies

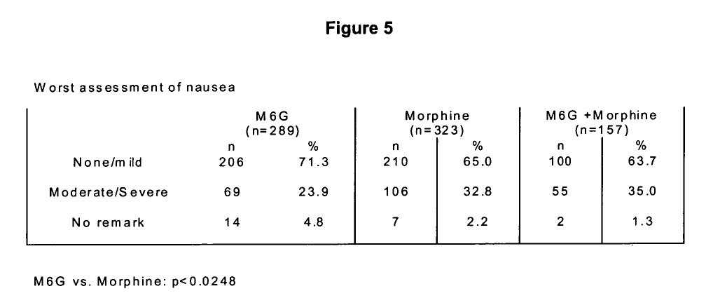

FIG. 6: Worst assessment of vomiting in the meta analysis of M6G clinical studies

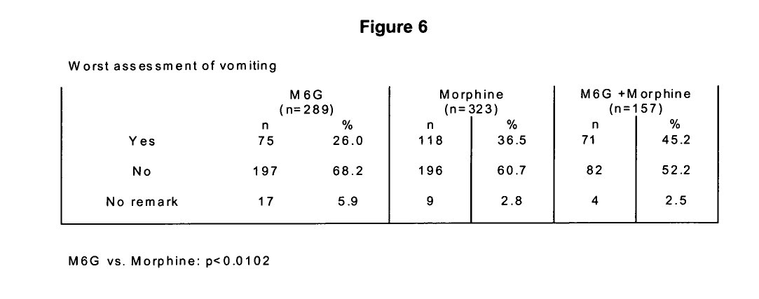

FIG. 7: Worst assessment of pain in the meta analysis of M6G clinical studies in the time window of 0-24 hours (A), 0-6 hours (B), and 6-24 hours (C).

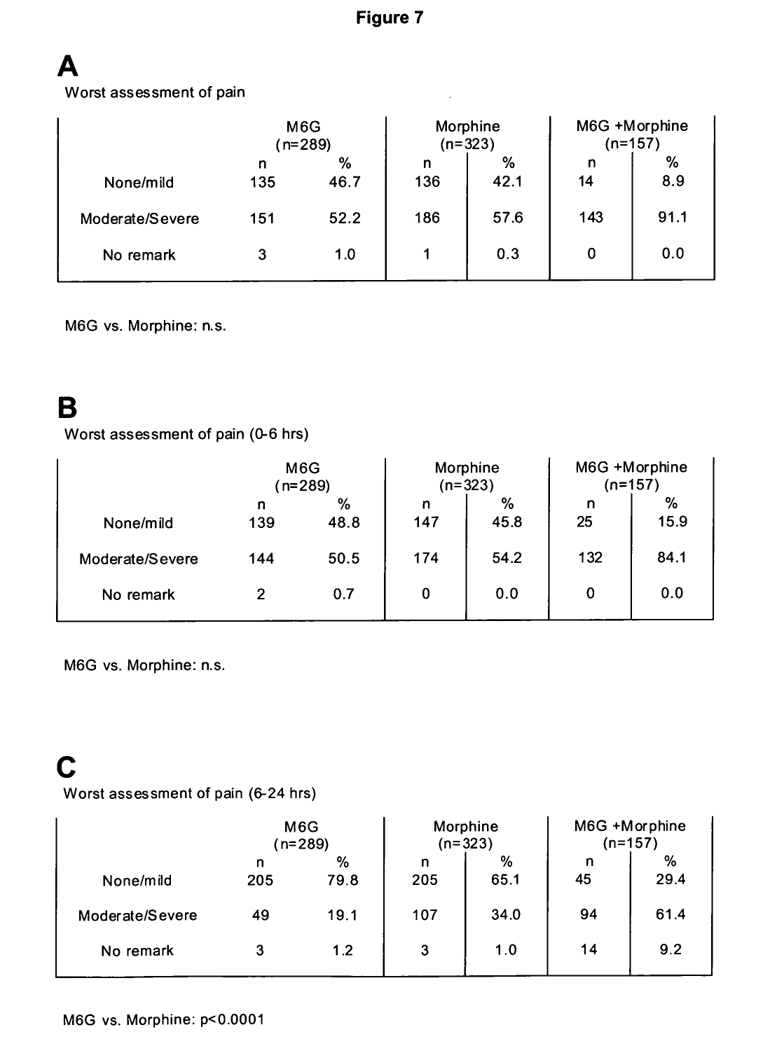

FIG. 8: Relationship between Concentration of M6G and Verbal Pain-Score (Subject 3043)

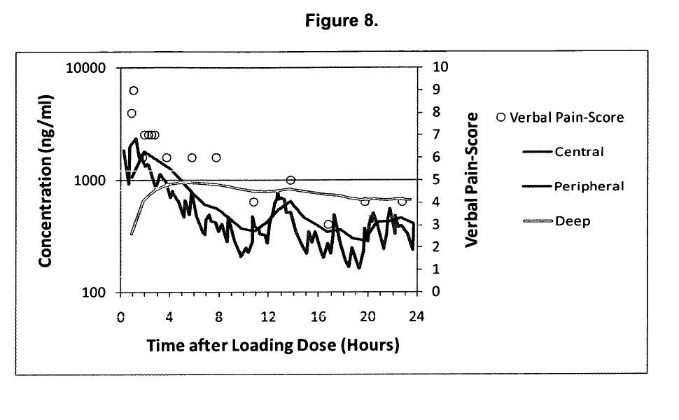

FIG. 9: PK/PD Modeling of M6G and Pain (Subject 3043)

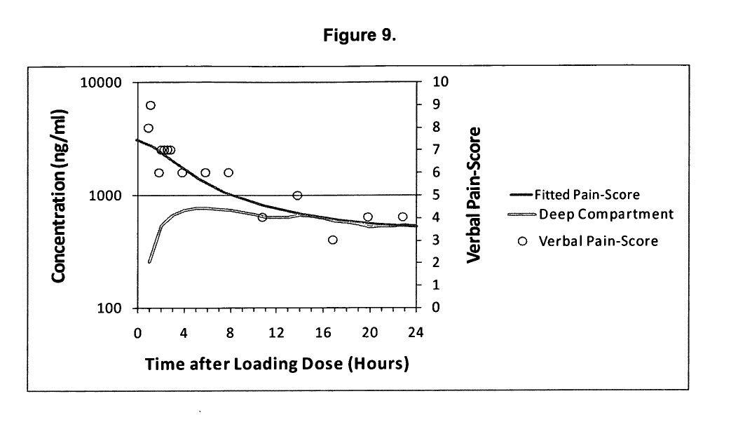

FIG. 10: PK/PD Modeling of M6G and Nausea/Vomiting

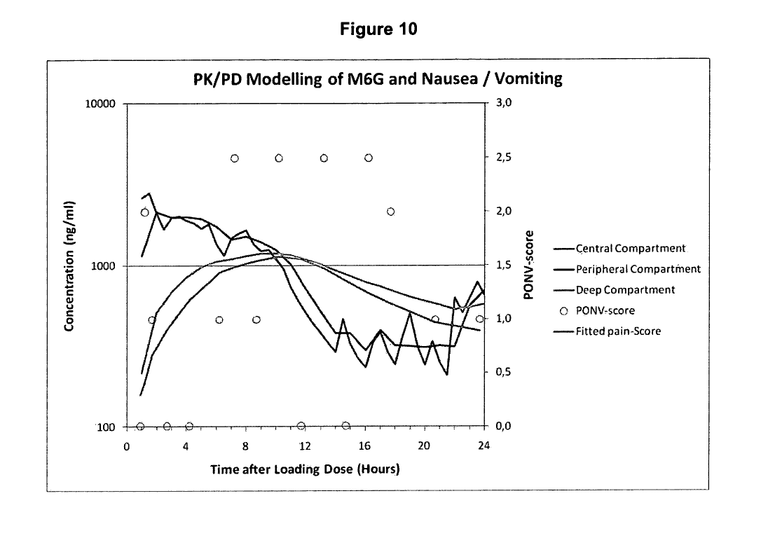

FIG. 11: Performance of Pain-Score Model for M6G: Goodness of Fit plot showing weighted residual (WRES) vs. subject number (M6GALLPD01)

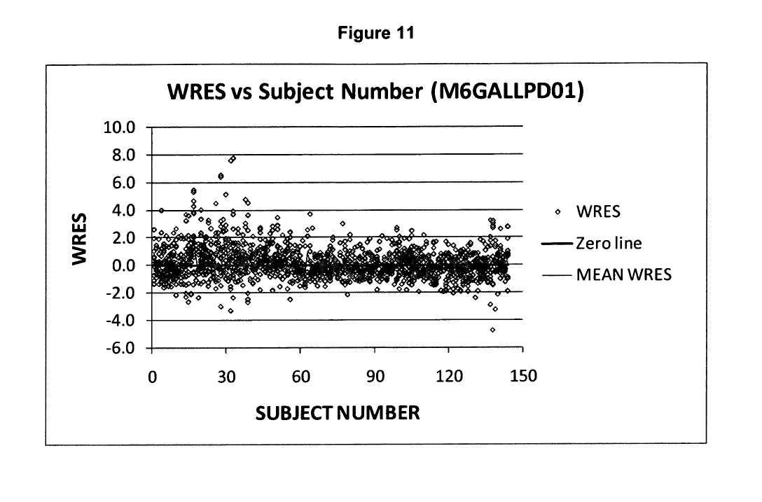

FIG. 12: Performance of Pain-Score Model for M6G: Goodness of Fit plot showing weighted residual (WRES) vs. subject number (M6GPONV01)

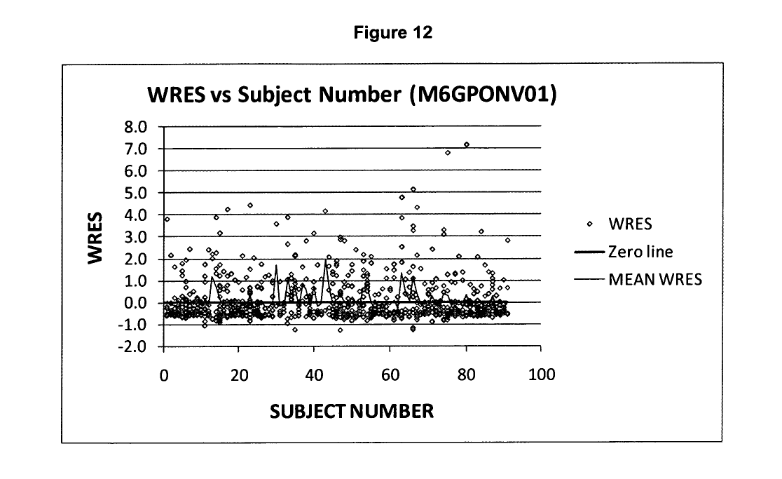

FIG. 13: Simulation of analgesic effect of 15 mg morphine vs. 30 mg and 45 mg M6G

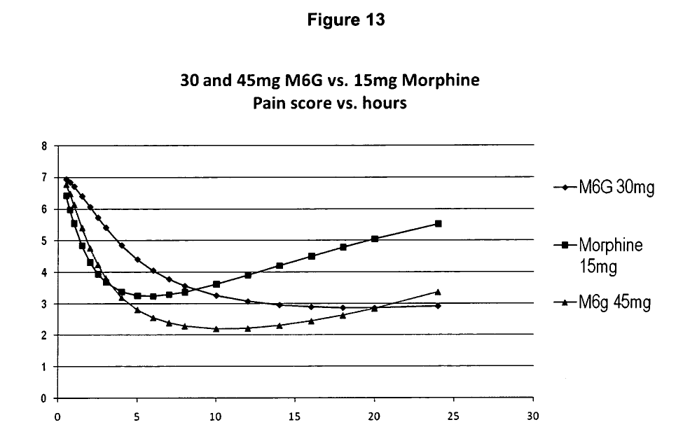

FIG. 14: Simulation of analgesic effect of 1.5 mg morphine vs. 45 mg M6G given 2 hours earlier than morphine

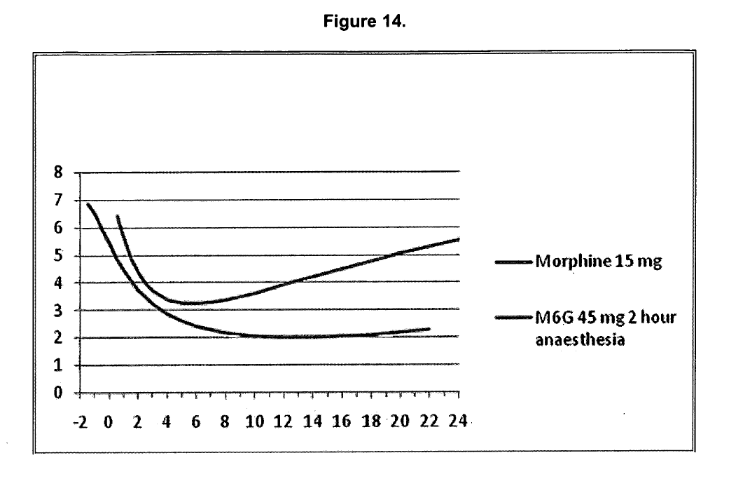

FIG. 15: Simulation of analgesic effect of a 45 mg starting dose combined with 1 to 3 subsequent doses of 15 mg M6G

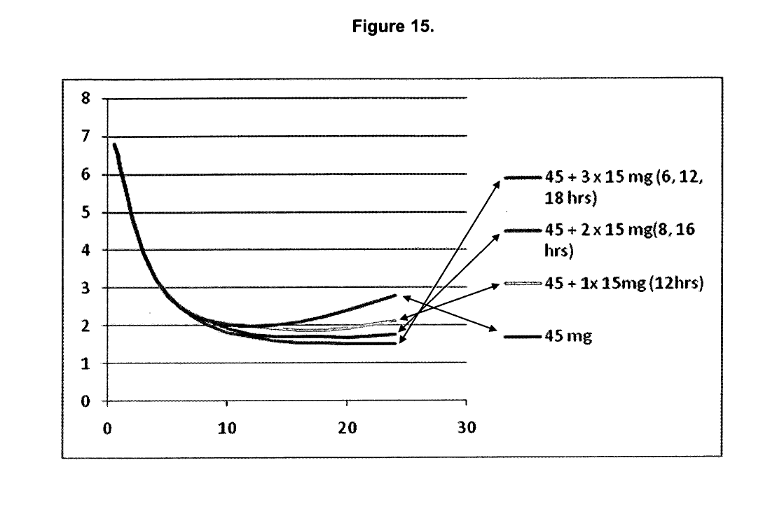

FIG. 16: Simulation of side effects (PONV) of 15 mg morphine vs. 30 mg and 45 mg M6G

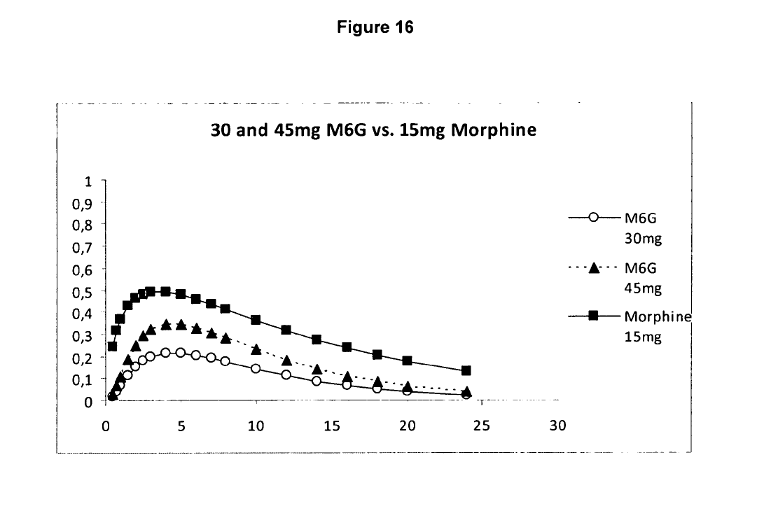

TABLE 1

| | M6G (n = 268) | Morphine (n = 249) |
|---|---|---|
| | n (%) | n (%) |
| Sex | | |
| Male | 83 (31.0%) | 81 (32.5%) |
| Female | 185 (69.0%) | 168 (67.5%) |
| Age (years) (mean (SD)) | 55.0 (12.74) | 54.3 (12.41) |
| Surgery | | |
| Total abdominal hysterectomy | 135 (50.4%) | 122 (49.0%) |
| Bowel/GI surgery | 83 (31.0%) | 85 (34.1%) |
| Major urological surgery | 50 (18.7%) | 42 (16.9%) |
| History of PONV | 95 (35.4%) | 83 (33.3%) |
| Previous motion sickness | 35 (13.1%) | 39 (15.7%) |
| Non-smoker | 139 (51.9%) | 145 (58.2%) |

TABLE 2

| Study Period | M6G | Morphine | Reduction | Significance |
|---|---|---|---|---|
| AUC 0-6 h | 1.35 | 1.85 | 27.0% | p = 0.047 |
| AUC 6-24 h | 5.32 | 7.44 | 28.5% | p = 0.018 |
| AUC 0-12 h | 3.19 | 4.33 | 26.3% | p = 0.048 |
| AUC 0-24 h | 7.34 | 9.70 | 24.3% | p = 0.027 |
| AUC 12-24 h | 3.92 | 5.19 | 24.5% | p = 0.045 |
| AUC 24-48 h | 9.21 | 10.27 | 10.3% | p = 0.504 |

TABLE 3

| Study No. | Population | n | M6G Doses |
|---|---|---|---|
| Phase II | | | |
| M6G001A | Hip replacement | 18 | 10, 15, 20 mg/70 kg with PCA |
| M6G012 | Hip replacement | 68 | 10, 20, 30 mg/70 kg |
| Phase III | | | |
| M6G015 | Knee replacement | 170 | 10, 20, 30 mg/70 kg |
| M6G022 | Major abdominal | 517 | 30 mg/70 kg, followed by PCA |
| | surgery | | |

TABLE 4

| | | Cmax | AUC0-∞ | t½ | CL | MRT0-∞ | Vss |
|---|---|---|---|---|---|---|---|
| | | (ng · mL − 1) | (ng · mL − 1 · h) | (h) | (mL · h − 1) | (h) | (mL) |
| 15 mg | n | 10 | 10 | 10 | 10 | 10 | 10 |
| | Mean | 1861 | 1930 | 1.478 | 7978 | 1.65 | 13013 |
| | SD | 673 | 311 | 0.220 | 1422 | 0.20 | 1657 |
| | Min | 1119 | 1392 | 1.130 | 6517 | 1.36 | 11229 |
| | Median | 1649 | 1885 | 1.560 | 7957 | 1.69 | 12558 |
| | Max | 2917 | 2302 | 1.783 | 10776 | 1.89 | 15979 |
| | Geometric | 1754 | 1906 | 1.462 | 7872 | 1.64 | 12923 |
| | Mean | | | | | | |
| 30 mg | n | 11 | 11 | 11 | 11 | 11 | 11 |
| | Mean | 3977 | 4186 | 1.723 | 7309 | 1.77 | 12806 |
| | SD | 942 | 612 | 0.296 | 1073 | 0.24 | 1606 |
| | Min | 2640 | 3358 | 1.223 | 5980 | 1.28 | 10448 |
| | Median | 4113 | 4000 | 1.811 | 7501 | 1.81 | 12188 |
| | Max | 5400 | 5017 | 2.024 | 8935 | 2.09 | 15949 |
| | Geometric | 3874 | 4145 | 1.698 | 7238 | 1.76 | 12717 |
| | Mean | | | | | | |
| 45 mg | n | 11 | 11 | 11 | 11 | 11 | 11 |
| | Mean | 5857 | 5906 | 1.641 | 7776 | 1.68 | 12863 |
| | SD | 1380 | 895 | 0.249 | 1143 | 0.23 | 1445 |
| | Min | 3988 | 4696 | 1.240 | 6178 | 1.23 | 10106 |
| | Median | 5856 | 5498 | 1.693 | 8184 | 1.78 | 13136 |
| | Max | 9233 | 7284 | 1.960 | 9584 | 1.95 | 15043 |
| | Geometric | 5724 | 5846 | 1.623 | 7698 | 1.66 | 12786 |
| | Mean | | | | | | |

TABLE 5

| | 15 mg/ | 30 mg/ | 45 mg/ | | |
|---|---|---|---|---|---|
| | 70 kg | 70 kg | 70 kg | | 90% CI |
| Parameter | Gear Geometric LS means | | | Slope | for Slope |
| Cₘₐₓ (ng · mL⁻¹) | 1745 | 3699 | 5724 | 1.082 | 0.966-1.198 |
| AUC₀₋ₜ (ng · mL⁻¹ · h) | 1765 | 3881 | 5751 | 1.080 | 1.039-1.120 |
| AUC_{0-∞} (ng · mL⁻¹ · h) | 1877 | 3967 | 5846 | 1.037 | 1.007-1.068 |

TABLE 6

| | | Cₘₐₓ | t_{1/2} | CL | AUC₀₋₆ | AUC_{0-∞} | Vss | MRT_{0-∞} |
|---|---|---|---|---|---|---|---|---|
| | | (ng · mL⁻¹) | (h) | (mL · h⁻¹) | (ng · mL⁻¹ · h) | (ng · mL⁻¹ · h) | (mL) | (h) |
| Dose 1-15 mg | n | 12 | 12 | 12 | 12 | 12 | 12 | 12 |
| | Mean | 1981 | 1.734 | 7169 | 1921 | 2126 | 14844 | 2.10 |
| | SD | 754 | 0.327 | 970 | 207 | 274 | 1977 | 0.36 |
| | Min | 979 | 1.414 | 5979 | 1580 | 1659 | 12279 | 1.56 |
| | Medan | 2089 | 1.647 | 6798 | 1951 | 2207 | 14228 | 2.17 |
| | Max | 2957 | 2.618 | 9041 | 2189 | 2509 | 19228 | 2.86 |
| | Geometric | 1837 | 1.710 | 7111 | 1911 | 2109 | 14732 | 2.07 |
| | Mean | | | | | | | |
| | | Cₘₐₓ | t_{1/2} | CLₛₛ | AUC₂₄₋₃₀ | AUC_{24-∞} | Vss | MRT_{24-∞} |
| | | (ng · mL⁻¹) | (h) | (mL · h⁻¹) | (ng · mL⁻¹ · h) | (ng · mL⁻¹ · h) | (mL) | (h) |
| Dose 5-15 mg | n | 12 | 12 | 12 | 12 | 12 | 12 | 12 |
| | Mean | 2369 | 1.496 | 7303 | 2078 | 2185 | 11986 | 1.65 |
| | SD | 627 | 0.190 | 820 | 229 | 254 | 1754 | 0.21 |
| | Min | 1289 | 1.190 | 6245 | 1745 | 1820 | 7830 | 1.20 |
| | Median | 2369 | 1.561 | 7173 | 2091 | 2213 | 12173 | 1.64 |
| | Max | 3361 | 1.794 | 8595 | 2402 | 2565 | 14062 | 1.92 |
| | Geometric | 2288 | 1.485 | 7261 | 2066 | 2171 | 11853 | 1.63 |
| | Mean | | | | | | | |

TABLE 7

| | M6G | Morphine | All |
|---|---|---|---|
| Total | 176/268 | 169/249 | 345/517 |
| Drop-outs (no PCA) | 14/23 | 4/10 | 20/33 |
| No PCA file | 6 | 6 | 12 |
| No Pain | 12 | 8 | 20 |
| No PONV | 65 | 65 | 130 |
| Pain | 144 | 151 | 295 |
| PONV | 91 | 94 | 185 |

TABLE 8

| | AAC (Pain) | AUC (PONV) | AAC/AUC |
|---|---|---|---|
| Morphine | | | |
| 10 mg | 45.7 | 4.8 | 9.46 |
| 15 mg | 64.6 | 7.4 | 8.73 |
| 20 mg | 79.4 | 9.9 | 8.03 |
| 25 mg | 91.0 | 12.3 | 7.43 |
| 30 mg | 102.7 | 14.5 | 7.09 |
| M6G | | | |
| 30 mg | 79.5 | 2.6 | 31.0 |
| 45 mg | 96.8 | 4.2 | 23.2 |
| 60 mg | 108.2 | 5.8 | 18.6 |
| 75 mg | 116.3 | 7.5 | 15.6 |
| 90 mg | 122.6 | 9.1 | 13.5 |

TABLE 9

| | EC 50 (ng/ml) | EC 50 (ng/ml) | EC 50 (PONV) |
|---|---|---|---|
| | (Pain) | (PONV) | EC 50 (pain) |
| Morphine | 21.8 | 32.7 | 1.5 |
| M6G | 177.0 | 1325.0 | 7.5 |

What is claimed is:

1-9. (canceled)

10. A method of treatment of post-operative pain in a patient, said method comprising:
providing an analgesically active polar metabolite of an opioid or the salts thereof in a pharmaceutically acceptable carrier to realize a medicament;
administering the medicament to the subject at least once before the end of a medical operation.

11. The method according to claim 10, wherein the medicament is administered to the patient at least once post-operatively.

12. The method according to claim 10, wherein the medicament is administered to the patient in a post-operative recovery phase.

13. The method according to claim 10, wherein the medicament is administered to the patient at least in a time frame selected from the group consisting of 10, 20, 30 45, 60, 90 and 120 minutes prior to a time the operation is terminated.

14. The method according to claim 10, wherein the medicament is administered to the patient pre-operatively.

15. The method according to claim 10, wherein the medicament is administered to the patient concomitantly with a induction of anesthesia.

16. The method according to claim 11, wherein the medicament is administered after a post-operative recovery phase.

17. The method according to claim 10, wherein the polar opioid metabolite is selected from the group consisting of morphine-6-glucuronide, morphine-3,6-diglucuronide, morphine-6-sulfate, 6-monoacetylmorphine, normorphine and morphine-3-ethereal sulfate.

18. The method according to claim 10, wherein the medicament is administered to the patient in an administration schedule of at least one of step a and step b as follows:
a) the medicament is administered at least one of: at least once pre-operatively and before a medical operation.
b) the medicament is administered at least one of: at least once during a post-operative recovery phase and to a maximum of about 48 hours after the end of the medical operation,
wherein administration is by perfusion or by patient-controlled-analgesia.

19. The method according to claim 10, wherein the active metabolite is M6G or its salts, in particular M6G hydrobromide, and the metabolite is administered at least about at least 10 minutes, at least 20 minutes, at least 30 minutes, at least 45 minutes or at least 60 minutes, 90 minutes or 120 minutes before the end of the operation with a dose of about 5 mg to 15 mg/70 kg body weight.

20. The method according to claim 19, wherein the dose is 10 mg/70 kg body weight.
